(19) United States
(12) Patent Application Publication
(10) Pub. No.: US 2026/0053788 A1
(43) Pub. Date: Feb. 26, 2026

(54) METHODS AND COMPOSITIONS FOR MODULATING HEMOSTASIS AND METABOLIC HOMEOSTASIS DURING HEMORRHAGE

(71) Applicant: Augusta University Research Institute, Inc., Augusta, GA (US)

(72) Inventor: Raghavan Pillai Raju, Augusta, GA (US)

(21) Appl. No.: 19/310,307

(22) Filed: Aug. 26, 2025

Related U.S. Application Data
(60) Provisional application No. 63/686,972, filed on Aug. 26, 2024.

(51) Int. Cl. A61K 31/455; A61K 31/195; A61P 7/04

(52) U.S. Cl. CPC A61K 31/455; A61K 31/195; A61P 7/04

(57) ABSTRACT

Therapeutic methods including the coadministration of niacin or niacin-related compound and tranexamic acid are provided. Typically, the treated subject have a hemorrhage (i.e., hemorrhagic injury) and may suffer from hemorrhagic shock. The methods typically include administering the subject an effective amount of niacin or niacin-related compound and tranexamic acid to reduce one or more symptoms of the hemorrhage. Symptoms include those associated with hemorrhagic injuries and shock such as death. The niacin or niacin-related compound and tranexamic acid can be administered in an effective amount to increase the subject's survival. Compositions and kits are also provided. The kits can include the niacin or niacin-related compound and tranexamic acid in the same or separate containers. In some forms, the kit includes a syringe, optionally an autoinjector syringe. For example, the kits can include an autoinjector syringe loaded with an effective amount of niacin or niacin-related compound and tranexamic acid.

### CROSS-REFERENCE TO RELATED APPLICATIONS

This application claims the benefit of and priority to U.S. Provisional Application No. 63/686,972, filed Aug. 26, 2024, which is hereby incorporated herein by reference in its entirety.

### STATEMENT REGARDING FEDERALLY SPONSORED RESEARCH

This invention was made with government support under W81XWH-22-1-0903/BA200322 awarded by the Department of Defense. The government has certain rights in the invention.

### FIELD OF THE INVENTION

The disclosed invention is generally directed to combination therapies for treatment of hemorrhagic injury.

### BACKGROUND OF THE INVENTION

Hemorrhagic shock (hemorrhagic injury or HI) is a life-threatening condition that occurs when the body loses a significant amount of blood, leading to inadequate tissue perfusion and oxygen delivery to vital organs. This can result in cellular and organ dysfunction, and if not treated promptly, it can lead to organ failure and death. Further, the body's cardiovascular system begins to shut down due to a large amount of blood loss. This loss of blood volume and oxygen-carrying capacity can lead to inadequate oxygen and nutrient delivery. As a result, mitochondria are no longer able to sustain aerobic metabolism for the production of oxygen and switch to the less efficient anaerobic metabolism to meet the cellular demand for adenosine triphosphate (Hooper, “Hemorrhagic Shock”, StatPearls, 2022).

Injury is the leading cause of death in the age-range 1-45 years in the general population and hemorrhage is a major preventable cause of mortality. In the management of hemorrhagic shock, the three important factors to be considered are hemostasis, volume replacement, and metabolic homeostasis. Though hemostasis may be attempted in the field, immediate volume replacement may not be always possible.

Thus, it is the object of the current invention to provide compositions and methods of treating Hemorrhagic shock (hemorrhagic injury or HI).

It is a further object of the invention to treat the shock in the field, particularly shortly after injury, for example at the scene of the injury and/or during transport to a medical facility.

### BRIEF SUMMARY OF THE INVENTION

Therapeutic methods including the coadministration of niacin or a niacin-related compound and tranexamic acid are provided. Typically, the treated subject has a hemorrhage (i.e., hemorrhagic injury) and may suffer from hemorrhagic shock. The methods typically include administering the subject an effective amount of niacin or a niacin-related compound and tranexamic acid to reduce one or more symptoms of the hemorrhage. Symptoms include those associated with hemorrhagic injuries and shock and included, but are not limited to, death, loss of homeostasis, rise in arterial blood pressure, rise in serum lactate level, and/or rise in blood pH. Thus, the niacin or niacin-related compound and tranexamic acid can be administered in an effective amount to increase survival of the subject.

The niacin or niacin-related compound and tranexamic acid can be administered in the same or different pharmaceutical compositions. The niacin or niacin-related compound and tranexamic acid can be administered by the same or different routes. Preferred routes are intravenous injection or infusion, and intramuscular injection. The niacin or niacin-related compound and tranexamic acid can be administered concomitantly, simultaneously, or sequentially. The niacin or niacin-related compound and tranexamic acid can be administered contemporaneously. In some forms, the niacin or niacin-related compound and tranexamic acid are administered between 0-30 minutes apart or any subrange or specific value there between. The niacin or niacin-related compound and tranexamic acid can be administered once, or multiple times, for example, two or more times 0.5-5 hours, preferably 1-3 hours, apart or any subrange or specific value there between. In some forms, the niacin or niacin-related compound is administered at a dosage of 2-15 mg/Kg body weight, the tranexamic acid is administered at a dosage of 5-100 mg/Kg body weight, or a combination thereof.

In some forms, administration of the combination of niacin or niacin-related compound and tranexamic acid achieves a result greater than administering to the subject niacin or niacin-related compound alone and/or tranexamic acid alone.

In some forms, administration of the combination of niacin or niacin-related compound and tranexamic acid achieves a result that is additive of the result obtained when administering to the subject niacin or niacin-related compound alone and tranexamic acid alone. Results include, but are not limited to, increased survival time and/or reduced, prevented, or reversed loss of homeosis optionally selected from reduced, prevented, and/or reversed a rise in mean arterial blood pressure; reduced, prevented, and/or reversed rise in serum lactate; reduced, prevented, and/or reversed rise blood pH; or a combination thereof.

In some forms, administration of the combination of niacin or niacin-related compound and tranexamic acid achieves a result that is more than additive of the result obtained when administering to the subject niacin or a niacin-related compound alone and tranexamic acid alone.

In some forms, the subject has a hemorrhagic injury and the niacin or niacin-related compound and tranexamic acid are administered to the subject at the scene of the injury. In some forms, the niacin or niacin-related compound and tranexamic acid are administered to the subject while transporting the subject to a medical center.

The niacin or niacin-related compound and tranexamic acid can be administered in further combination with one or more other treatments. The additional treatment can be, for example, one or more of controlling bleeding, replacing fluids, administering other medications optionally selected from vasopressors, such as norepinephrine or vasopressin, to increase blood pressure, etc.

Compositions for use in the disclosed methods and use thereof are also provided. The compositions are typically pharmaceutical compositions including one or both of niacin or a niacin-related compound and tranexamic acid, and pharmaceutically acceptable carrier.

Kits including the compositions are also provided. The kits can include the niacin or niacin-related compound and tranexamic acid in the same or separate containers. In some forms, the kit includes a syringe, optionally an autoinjector syringe. In a particular form, the kits include an autoinjector syringe loaded with an effective amount of niacin or a niacin-related compound and tranexamic acid.

### BRIEF DESCRIPTION OF THE DRAWINGS

FIG. 1 shows TXA+NAC improves survival in uncontrolled hemorrhage; TXA (100 mg/Kg), NAC (10 mg/Kg). Control (Veh) vs TXA+NAC p<0.0001; Veh vs NAC, p<0.05; veh vs TXA, ns; TXA vs NAC, ns; NAC vs NAC+TXA, p<0.05; TXA vs NAC+TXA, p<0.01. n=9 rats/group.

FIG. 2 shows mean duration of survival in an uncontrolled hemorrhage model treated with TXA, NAC or NAC+TXA. n=9/group.

FIG. 3 shows mean arterial pressure (MAP) at 30-60 minutes after liver injury. n=4-5

FIGS. 4A-4B show blood lactate (FIG. 4A) and blood pH (FIG. 4B) with or without a combinatorial treatment of TXA (100 mg/kg) and Niacin (10 mg/kg), post liver injury (n=3).

FIG. 5 is a Kaplan-Meier survival curve for uncontrolled hemorrhage without fluid resuscitation with or without the intramuscular administration of a combination of Niacin and Tranexamic acid (NAC—10 mg/kg+TXA—100 mg/kg, n=4).

FIG. 6 is a bar graph showing Survival duration of each group of rats shown in FIG. 5.

### DETAILED DESCRIPTION OF THE INVENTION

### A. Definitions

As used herein, the terms “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

As used herein, the term “dosage regime” refers to drug administration regarding formulation, route of administration, drug dose, dosing interval and treatment duration.

As used herein, the terms “individual”, “host”, “subject”, and “patient” are used interchangeably, and refer to a mammal, including, but not limited to, primates, for example, human beings, as well as rodents, such as mice and rats, and other laboratory animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect.

The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

“Treatment” or “treating” means to administer a composition to a subject or a system with an undesired condition (e.g., hemorrhagic shock or hemorrhagic injury). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

“Prevention” or “preventing” means to administer a composition to a subject or a system at risk for an undesired condition. The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, the compositions may inhibit or reduce the activity and/or quantity of one or more disclosed mechanisms, pathways, or symptoms by about 10%, 20%, 30%, 40%, 50%, 75%, 85%, 90%, 95%, or 99% from the activity and/or quantity of the same inhibitor in subjects that did not receive or were not treated with the compositions. In some forms, the inhibition and reduction are compared according to the level of mRNAs, proteins, cells, tissues, and organs.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### B. Compositions

Compositions for use in combination therapies for treating hemorrhagic shock are provided. The combination therapies include administration of an effective amount of niacin (NAC) or a niacin-related compound in combination with tranexamic acid (TXA) to a subject in need thereof.

### 1. Active Agents

i. Niacin (NAC) and Niacin-Related Compounds

The combination therapies include niacin itself or a pharmaceutically acceptable salt thereof (also referred to herein as NAC and niacin), or a derivative or metabolite thereof or other niacin receptor ligand (collectively referred to as “niacin-related compounds”), Niacin potentiates mitochondrial function and reduces inflammation and extends survival following hemorrhagic shock, when given intravenously. Niacin (Vitamin B3) is a precursor to NAD+ (nicotinamide adenine dinucleotide), a coenzyme in cellular metabolism. NAD+ is essential for mitochondrial function as it plays a key role in the electron transport chain, which generates ATP. By increasing NAD+ levels, niacin enhances mitochondrial efficiency and energy production (Pirinen et al., Cell Metabolism, 31(6), 2020). Niacin exerts anti-inflammatory effects by modulating immune responses and inhibiting pro-inflammatory pathways. Additionally, niacin's role in increasing NAD+ also contributes to its anti-inflammatory properties by supporting cellular repair mechanisms and reducing oxidative stress, which is often linked to inflammation.

Niacin derivatives and metabolites include nicotinamide, nicotinamide riboside, and nicotinamide mononucleotide.

Niacin binds to GPR109a (also called niacin receptor) which is a receptor for beta-hydroxybutyric acid. Thus, an example of a niacin receptor ligand other than NAC is beta-hydroxybutyric acid.

ii. Tranexamic Acid (TXA)

The combination therapies include tranexamic acid itself or a pharmaceutically acceptable salt thereof (also referred to therein as TXA or tranexamic acid). In animal models and in the humans, it has been shown that tranexamic acid (TXA) improves hemostasis. TXA is a synthetic reversible competitive inhibitor to the lysine receptor found on plasminogen. The binding of this receptor prevents plasmin (activated form of plasminogen) from binding to and ultimately stabilizing the fibrin matrix. As a result, inhibition of plasminogen activation results in stabilization of the preformed fibrin meshwork produced by secondary hemostasis. Antifibrinolytic agents including tranexamic acid (TXA) have been shown to be effective at preventing bleeding complications in a variety of hemostatic challenges and reduce mortality with minimal adverse effects in some settings (Cai et al., Eur J Haematol., 2020 February; 104(2): 79-87).

iii. Additional Active Agents

In some embodiments, the combination therapy includes additional active agents. In addition to niacin or a niacin-related compound and tranexamic acid, the combination therapies can include any of the additional agents or components discussed herein, or known in the art to be co-administered with niacin or a niacin-related compound and tranexamic acid, to treat hemorrhagic shock. For example, agents that help in formation of blood clots such as but not limited to Epsilon-Aminocaproic Acid (EACA) or Aprotinin or Desmopressin (DDAVP) can be administered in combination with niacin or niacin-related compound and tranexamic acid.

### 2. Formulations

Formulations of and pharmaceutical compositions including one or more active agents are provided. The combination therapies can include administration of the active agents together in the same admixture, or in separate admixtures. Therefore, the pharmaceutical compositions can include niacin or a niacin-related compound and tranexamic acid in combination with any additional active agents. In some embodiments, the pharmaceutical compositions can include one or more additional active agents. Therefore, in some embodiments, the pharmaceutical composition includes two, three, or more active agents. The pharmaceutical compositions can be formulated as a pharmaceutical dosage unit, referred to as a unit dosage form. Such formulations typically include an effective amount of niacin or a niacin-related compound, tranexamic acid, or both. Effective amounts of the disclosed active agents are discussed in more detail below. It will be appreciated that in some embodiments the effective amount of niacin or niacin-related compound and tranexamic acid or other active agents in a combination therapy is different from that amount that would be effective when administered individually. For example, in some embodiments the effective amount of niacin or niacin-related compound and/or tranexamic acid is a lower dosage of the niacin or niacin-related compound and/or tranexamic acid in a combination therapy than the dosage of the niacin or niacin-related compound and/or tranexamic acid that is effective when one agent is administered without the other. Alternatively, in some embodiments the effective amount of niacin or niacin-related compound and/or tranexamic acid, is a higher dosage of the niacin or niacin-related compound and/or tranexamic acid in a combination therapy than the dosage of the niacin or niacin-related compound and/or tranexamic acid that is effective when one agent is administered without the other. In other embodiments, the dosage of one agent is higher and the dosage of the other agent is lower than one agent is administered without the other. In some cases, the agents are not effective when administered alone, and only effective when administered in combination.

i. Delivery Vehicles

The active agents can be administered and taken up into the cells of a subject with or without the aid of a delivery vehicle. Appropriate delivery vehicles for the disclosed active agents are known in the art and can be selected to suit the particular agent. For example, in some embodiments, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

The active agent(s) can be incorporated into a delivery vehicle prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C. The release point and/or period of release can be varied as discussed above.

ii. Pharmaceutical Compositions

Pharmaceutical compositions including active agent(s) with or without a delivery vehicle are provided. Pharmaceutical compositions can be for administration by parenteral (intramuscular, intraperitoneal, intravenous (IV) or subcutaneous injection), enteral, or transmucosal (nasal, vaginal, rectal, or sublingual) routes of administration or using bioerodible inserts and can be formulated in dosage forms appropriate for each route of administration.

In certain embodiments, the compositions are administered locally, for example, by injection directly into a site to be treated. In some embodiments, the compositions are injected or otherwise administered directly into the vasculature onto vascular tissue at or adjacent to the intended site of treatment. Typically, local administration causes an increased localized concentration of the compositions which is greater than that which can be achieved by systemic administration. Targeting of the molecules or formulation can be used to achieve more selective delivery.

a. Formulations for Parenteral Administration

Active agent(s) and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

b. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form including single or multiple units. Examples of multiple units include, but are not limited to, multilayer tablets and, capsules containing tablets, beads, or granules etc. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

In one embodiment, the compounds are formulated for pulmonary delivery, such as intranasal administration or oral inhalation. The respiratory tract is the structure involved in the exchange of gases between the atmosphere and the blood stream. Pulmonary administration of therapeutic compositions including low molecular weight drugs has been observed, for example, beta-androgenic antagonists to treat asthma. Other therapeutic agents that are active in the lungs have been administered systemically and targeted via pulmonary absorption. The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e., non-aqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

### 3. Adjunct and Additional Therapies and Procedures

The combination therapies can be administered to a subject in combination with one or more adjunct therapies or procedures, or can be an adjunct therapy to one or more primary therapies or producers. The additional therapy or procedure can be simultaneous or sequential with the combination therapy. In some embodiment the additional therapy is performed between drug cycles or during a drug holiday that is part of the combination therapy dosage regime. In preferred embodiment, the additional therapy is a conventional treatment for hemorrhagic shock (hemorrhagic injury or HI).

### C. Methods of Treatment

Methods of treating a subject in need thereof are provided. In some forms, the method includes using a combination of niacin or a niacin-related compound and tranexamic acid to treat hemorrhagic shock and/or hemorrhagic injury (HI) in a subject. In some forms, the method includes using a combination of niacin or a niacin-related compound and tranexamic acid with other active agents to treat the hemorrhagic shock and/or injury in a subject.

In certain forms, the methods include administering to a subject an effective amount of niacin or a niacin-related compound and tranexamic acid or a pharmacologically active salt thereof in combination with one or more active agents to reduce or treat hemorrhagic shock and/or injury.

device.

### 1. Subjects to be Treated

Subjects or patients for treatment with a combination of niacin or a niacin-related compound and tranexamic acid are described.

In some forms, the subject is has a non-compressible hemorrhage, compressible hemorrhage, or both. The methods can thus be used to treat hemorrhagic shock, hemorrhagic injury, or the combination thereof.

In some forms, the subject is a trauma patient with significant hemorrhage or those at high risk of developing severe bleeding. In some forms, the subject is a surgical patient with a high risk of bleeding, such as those undergoing cardiac, cardiothoracic, orthopedic, gynecological, and liver surgeries. Patients undergoing major surgeries are at risk of significant blood loss or those with a history of heavy bleeding during surgery.

In some forms, the subject is an obstetric patient indicated for the management of postpartum hemorrhage (PPH), particularly when there is ongoing blood loss despite standard uterotonic therapy. In some forms, the subject is a woman with known risk factors for PPH, such as multiple gestations, large babies, or a history of PPH. In some forms, the subject is a woman with heavy menstrual bleeding (menorrhagia).

In some forms, the subject has a bleeding disorder such as hemophilia, Von Willebrand disease, Factor VII deficiency, Factor XI deficiency, excessive bleeding during minor surgical procedures, or any platelet function disorder. In some forms, the subject has one or more of gunshot wound(s), stab wound(s), deep wound(s) optionally that reach major arteries or veins, severe blunt trauma such as from car accidents, falls, or crush injuries which can cause internal bleeding, fractures associated with significant internal bleeding, amputations that result in major blood loss, dental work, or epistaxis.

In some forms, the subjects are soldiers wounded in combat.

The compositions and methods can be used at the scene of injury (e.g., in the field) and/or during transportation to a medical treatment facility (e.g., clinic or hospital) and/or after arrival at a medical treatment facility (e.g., clinic or hospital).

### 2. Symptoms to be Treated

The disclosed compositions and methods can be used to reduce death and other symptoms associated with hemorrhagic shock and injury. Thus, in some forms the symptom is loss or reduction in homeostasis and/or death. In some forms, a combination of niacin or niacin-related compound and tranexamic acid can be used to treat a subject displaying one or more other symptoms of hemorrhagic shock or injury such as tachycardia (rapid heart rate), hypotension, pale, cool, and clammy skin caused by vasoconstriction and blood being redirected to vital organs, weak or thready pulse reflecting decreased cardiac output, rapid, shallow breathing caused by the body's attempts to compensate for low oxygen levels, altered mental status including confusion, anxiety, or loss of consciousness due to inadequate brain perfusion, and decreased urine output caused by kidney perfusion issues. Other symptoms include blue lips or fingernails, excessive sweating, abdominal pain and swelling, blood in the urine or stool, vaginal bleeding (heavy, not normal menstruation), vomiting blood, or chest pain or shortness of breath.

In some forms the compositions and methods improve or reduce loss of homeosis. For example, in some embodiments, the compositions and methods reduce, prevent, and/or reverse a rise in mean arterial blood pressure; reduce, prevent, and/or reverse a rise in serum lactate; reduce, prevent, and/or reverse a rise blood pH; or a combination thereof.

### 3. Methods of Administration and Dosage Regimes

The combination therapies and treatment regimens typically include treatment of a disease or symptom thereof, or a method for achieving a desired physiological change, including administering to an animal, such as a mammal, especially a human being, an effective amount of niacin or a niacin-related compound and tranexamic acid to treat the disease or symptom thereof, or to produce the physiological change, wherein the chemical agents or components are administered together, such as part of the same composition, or administered separately and independently at the same time or at different times (i.e., administration of niacin or niacin-related compound and tranexamic acid is separated by a finite period of time from each other). Therefore, the term “combination” or “combined” is used to refer to either concomitant, simultaneous, or sequential administration of the niacin or a niacin-related compound and tranexamic acid. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

In preferred embodiments, administration of the niacin or niacin-related compound and tranexamic acid achieves a result greater than when either the niacin or niacin-related compound or tranexamic acid is administered alone or in isolation. For example, in some embodiments, the result achieved by the combination is partially or completely additive of the results achieved by the individual components alone. In the most preferred embodiments, the result achieved by the combination is more than additive of the results achieved by the individual components alone. In some embodiments, the effective amount of one or both agents used in combination is lower than the effective amount of each agent when administered separately. In some embodiments, the amount of one or both agents when used in the combination therapy is sub-therapeutic when used alone.

A treatment regimen of the combination therapy can include one or multiple administrations of niacin or a niacin-related compound and tranexamic acid. In certain embodiments, niacin or niacin-related compound can be administered simultaneously with tranexamic acid. Where niacin or niacin-related compound and tranexamic acid are administered at the same time, the niacin or niacin-related compound and tranexamic acid can be, but need not necessarily be, in the same pharmaceutical composition.

In exemplary forms, niacin or niacin-related compound and tranexamic acid are administered once, together in the same or separate admixture. In other forms, niacin or niacin-related compound and tranexamic acid are administered together in the same or separate admixture two, three, four, five, or more times every 1-3 hours, or any discrete time or subrange of times therebetween.

In some embodiments niacin or a niacin-related compound and tranexamic acid are administered sequentially, for example, in two or more different pharmaceutical compositions. In certain embodiments, the niacin or niacin-related compound is administered prior to the first administration of the tranexamic acid. In other embodiments, the tranexamic acid is administered prior to the first administration of the niacin or niacin-related compound. For example, the niacin or niacin-related compound and tranexamic acid can be administered to a subject at the same time.

Alternatively, the niacin or niacin-related compound and tranexamic acid are administered to the subject minutes or hours apart. For example, the niacin or niacin-related compound can be administered at least 1, 2, 3, 5, 10, 15, 20, 24 or 30 minutes or hours prior to or after administering of the tranexamic acid. Alternatively, the tranexamic acid can be administered at least 1, 2, 3, 5, 10, 15, 20, 24 or 30 minutes or hours prior to or after administering of the niacin or niacin-related compound. In certain embodiments, additive or more than additive effects of the administration of niacin or niacin-related compound in combination with tranexamic acid is evident after 0.25 hours to 24 hours following administration, or any subrange or specific number therebetween.

Dosage regimens or cycles of the agents are also provided and can be completely or partially overlapping, or can be sequential. For example, in some embodiments, all such administration(s) of the niacin or niacin-related compound occur before or after administration of the tranexamic acid. Alternatively, administration of one or more doses of the niacin or niacin-related compound can be temporally staggered with the administration of tranexamic acid to form a uniform or non-uniform course of treatment whereby one or more doses of niacin or niacin-related compound are administered, followed by one or more doses of tranexamic acid, followed by one or more doses of niacin or niacin-related compound; or one or more doses of tranexamic acid are administered, followed by one or more doses of niacin or niacin-related compound, followed by one or more doses of tranexamic acid; etc., all according to whatever schedule is selected or desired by the researcher or clinician administering the therapy.

An effective amount of each of the agents can be administered as a single unit dosage (e.g., as dosage unit), or sub-therapeutic doses that are administered over a finite time interval. Such unit doses may be administered on an hourly or hoursly or daily basis for a finite time period, such as up to 12 hours, 1 day, 18 hours, 2 days, 3 days, or up to 5 days, etc., are all specifically contemplated.

In some forms dosage of tranexamic acid can range from 4 mg/kg-110 mg/kg body weight, 3 mg/kg-120 mg/kg body weight, 2 mg/kg-130 mg/kg body weight, 1 mg/kg-140 mg/kg body weight, 10 mg/kg-90 mg/kg body weight, 15 mg/kg-80 mg/kg body weight, 20 mg/kg-70 mg/kg body weight, preferably 5 mg/Kg body weight-100 mg/Kg body weight.

In some forms dosage of niacin or niacin-related compound can range from 1 mg/kg-20 mg/kg body weight, 0.5 mg/kg-25 mg/kg body weight, 5 mg/kg-15 mg/kg body weight, 8 mg/kg-12 mg/kg body weight, preferably 2 mg/kg body weight-15 mg/Kg body weight.

### D. Higher Order Combination Therapies

The disclosed methods for treating hemorrhagic shock (hemorrhagic injury or HI) can be used in combination with other active agents. For example, agents can include those that help in formation of blood clots such as but not limited to Epsilon-Aminocaproic Acid (EACA) or Aprotinin or Desmopressin (DDAVP) can be administered in combination with niacin or niacin-related compound and tranexamic acid.

As used herein, “higher order combination” or “combined” refer to either concomitant, simultaneous, or sequential administration of niacin or niacin-related compound and tranexamic acid with one or more additional therapeutics.

In some forms, the niacin or niacin-related compound and tranexamic acid and other therapeutic agents are administered separately through the same route of administration. In other forms, the niacin or niacin-related compound and tranexamic acid and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

The niacin or niacin-related compound and tranexamic acid can be administered before the additional treatment, concurrently with the treatment, post-treatment, or during remission of the shock. When administered in combination, the niacin or niacin-related compound and tranexamic acid and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

Examples of preferred additional therapeutic agents include other conventional therapies known in the art for treating the hemorrhagic shock.

### E. Kits

Medical kits are also disclosed. The medical kits can include, for example, a dosage supply of the niacin or niacin-related compound or tranexamic acid, or a combination thereof, in separately or together in the same admixture. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. In a particular form, the device for administration is an autoinjector. The kits can include printed instructions for administering the compound in a use as described above.

The disclosed invention can be further understood by the following numbered paragraphs.

1. A method of treating a subject with a hemorrhage including administering the subject an effective amount of niacin or niacin-related compound and tranexamic acid to reduce one or more symptoms of the hemorrhage.

2. A method of treating a subject with hemorrhagic shock including administering the subject an effective amount of niacin or niacin-related compound and tranexamic acid to reduce one or more symptoms of the hemorrhage.

3. The method of paragraphs 1 or 2 wherein the one or more symptoms include death, loss of homeostasis, rise in arterial blood pressure, rise in serum lactate level, and/or rise in blood pH.

4. The method of any one of paragraphs 1-3 wherein the niacin or niacin-related compound and tranexamic acid are administered in the same pharmaceutical composition.

5. The method of any one of paragraphs 1-3, wherein the niacin or niacin-related compound and tranexamic acid are in different pharmaceutical compositions.

6. The method of any one of paragraphs 1-5, wherein the niacin or niacin-related compound and tranexamic acid are administered by the same route.

7. The method of paragraph 5, wherein the niacin or niacin-related compound and tranexamic acid are administered by different routes.

8. The method of any one of paragraphs 1-7, wherein the niacin or niacin-related compound and tranexamic acid are administered concomitantly, simultaneously, or sequentially.

9. The method of any one of paragraphs 1-8, wherein niacin or niacin-related compound and tranexamic acid are administered contemporaneously.

10. The method of any one of paragraphs 1-9, wherein the niacin or niacin-related compound and tranexamic acid are administered between 0-30 minutes apart or any subrange or specific value there between.

11. The method of any one of paragraphs 1-10, wherein the niacin or niacin-related compound and tranexamic acid are administered once.

12. The method of any one of paragraphs 1-11, wherein the niacin or niacin-related compound and tranexamic acid are administered two or more times 0.5-5 hours, preferably 1-3 hours, apart or any subrange or specific value there between.

13. The method of any one of paragraphs 1-12, wherein niacin or niacin-related compound is administered at a dosage of 2-15 mg/Kg body weight.

14. The method of any one of paragraphs 1-13, wherein tranexamic acid is administered at a dosage of 5-100 mg/Kg body weight.

15. The method of any one of paragraphs 1-14, wherein the niacin or niacin-related compound and/or tranexamic acid are administered by intravenously (e.g., bolus injection) or infusion.

16. The method of any one of paragraphs 1-15, wherein the niacin or niacin-related compound and/or tranexamic acid are administered by intramuscular injection.

17. The method of any one of paragraphs 1-16, wherein administration of the combination of niacin or niacin-related compound and tranexamic acid achieves a result greater than administering to the subject niacin or niacin-related compound alone and/or tranexamic acid alone.

18. The method of any one of paragraphs 1-17, wherein administration of the combination of niacin or niacin-related compound and tranexamic acid achieves a result that is additive of the result obtained when administering to the subject niacin or niacin-related compound alone and tranexamic acid alone.

19. The method of any one of paragraphs 1-18, wherein administration of the combination of niacin or niacin-related compound and tranexamic acid achieves a result that is more than additive of the result obtained when administering to the subject niacin or niacin-related compound alone and tranexamic acid alone.

20. The method of any one of paragraphs 17-19, wherein the result is increased survival time and/or reduced, prevented, or reversed loss of homeosis optionally selected from reduced, prevented, and/or reversed a rise in mean arterial blood pressure; reduced, prevented, and/or reversed rise in serum lactate; reduced, prevented, and/or reversed rise blood pH; or a combination thereof.

21. The method of any one of paragraphs 1-20, wherein the subject has a hemorrhagic injury and the niacin or niacin-related compound and tranexamic acid are administered to the subject at the scene of the injury.

22. The method of any one of paragraphs 1-21, wherein the niacin or niacin-related compound and tranexamic acid are administered to the subject while transporting the subject to a medical center.

23. The method of any one of paragraphs 1-22, wherein niacin or niacin-related compound and tranexamic acid are administered in further combination with one or more other treatments.

24. The method of paragraph 23, wherein the additional treatment is one or more of controlling bleeding, replacing fluids, administering other medications optionally selected from vasopressors, such as norepinephrine or vasopressin, to increase blood pressure.

25. A composition for use in the method of any one of paragraphs 1-24.

26. Use of the composition of paragraph 25 in the method of any one of paragraphs 1-25.

27. A kit including the composition of paragraph 25.

28. The kit of paragraph 27, wherein the niacin or niacin-related compound and tranexamic acid are in the same or separate containers.

29. The kit of paragraphs 27 and 28, including an syringe, optionally an autoinjector syringe.

30. The kit of any one of paragraphs 27-29, including an autoinjector syringe loaded with an effective amount of niacin or niacin-related compound and tranexamic acid.

31. Any of foregoing methods wherein the subject is not currently treated and/or the results are independent of fluid resuscitation.

32. A composition, method, use, or kit as described herein, optionally in more of the text, including the “examples”, and the figures.

### EXAMPLES

### Example 1: Niacin (NAC)+Tranexamic Acid (TXA) Improves Survival in Uncontrolled Hemorrhage without Fluid Resuscitation

### Method

Male Sprague Dawley rats were obtained from Charles River Laboratory (Wilmington, MA, USA), and were housed in the Augusta University animal facility. The animals were anesthetized with 2.5% isoflurane. Two femoral arteries and one femoral vein were cannulated using PE-50 tubing. One of the arteries was connected to a blood pressure analyzer for the real-time monitoring of mean arterial pressure (MAP), while the other artery was used for bleeding. A midline laparotomy was performed to expose the abdominal cavity. Approximately 65% of the left lateral and medial lobes of the liver were resected to cause bleeding and injury, and following this, the incision was closed aseptically in two layers. Animals were given NAC (10 mg/Kg), TXA (100 mg/Kg), or NAC (10 mg/Kg)+TXA (100 mg/Kg) or vehicle intravenously through the femoral vein after the liver resection, and survival was monitored. Blood gas values were measured at the end of the experiment (FIGS. 4A-4B).

### Results

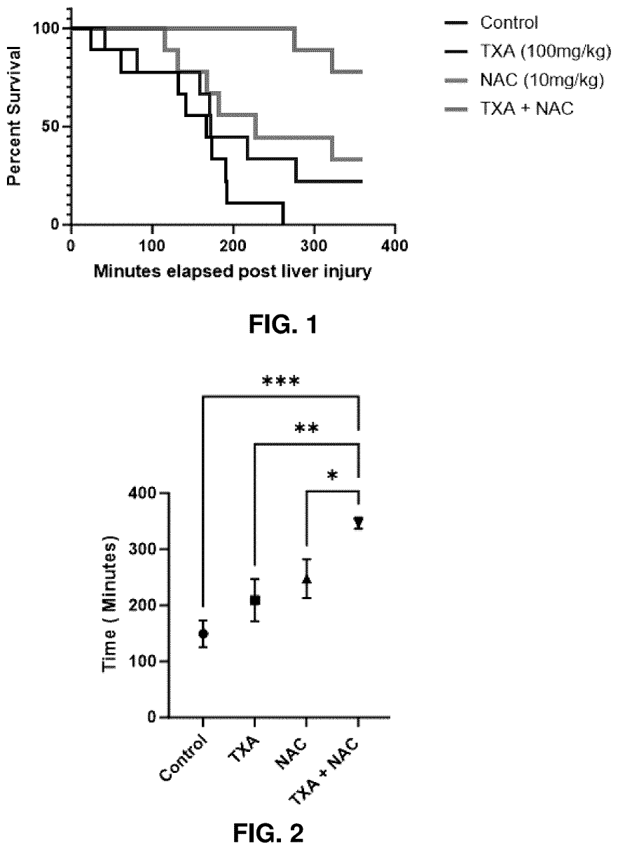

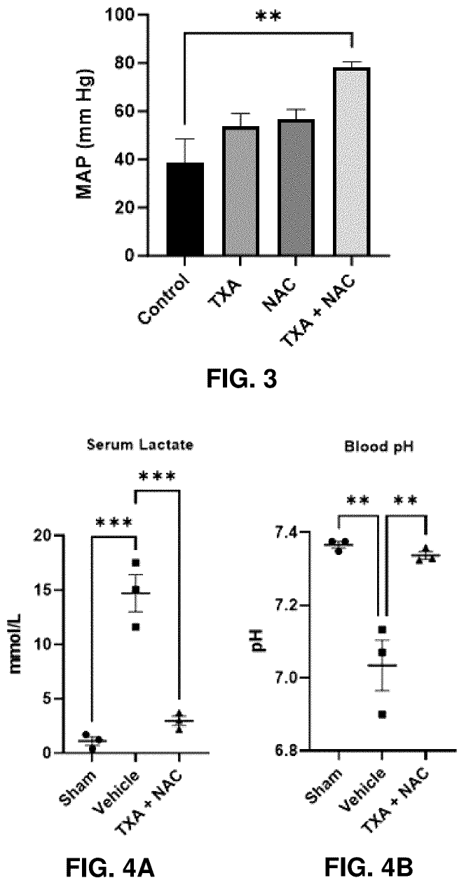

It was challenging to develop an uncontrolled hemorrhage model in the rat by liver resection method. Initial attempts to resect only the left lateral lobe was not successful as the rats survived this procedure. The modified procedure was used by resecting 65% of the left lateral and medial lobes, after approval from IACUC and ACURO. FIG. 1 shows the results of the completed Task. As shown clearly, after resection of lateral and medial lobes, most of the rats died within 3 hours whereas some of the rats that received NAC or NAC+TXA survived for the entire 6 hour duration of the study. The statistical significance of each of the survival curves is provided in the legend to FIG. 1. When mean survival duration was analyzed, vehicle treated rats survived for an average duration of 150 minutes while the rats treated with NAC+TXA survived for about 350 minutes (FIG. 2). It is likely that some of the rats that received NAC+TXA and few that received NAC alone could have survived longer than 6 hours. Mean MAP was analyzed between 30-60 minutes in each animal after the injury, and as represented in FIG. 3, NAC+TXA significantly improved MAP after liver resection. The results are promising for use of this formulation for pre-hospital care.

The impact of this treatment may include reducing the incidence of death due to hemorrhagic injury and shock by establishing the salutary effect of NAC-TXA (niacin-tranexamic acid combination). NAC-TXA modulates metabolic homeostasis and hemostasis simultaneously following severe blood loss. Use include enhancing survival and recovery from combat-related injury in current and future operational scenarios. Injury is the leading cause of death in the age-range 1-45 years in the general population and hemorrhage is a major preventable cause of mortality. The techniques, strategies and knowledge developed herein are expected to benefit the military and general public. Overall the significance of the project is very high considering the lack of availability of well-tested agents that can prolong life following severe hemorrhage. Furthermore the data presented herein shows the beneficial effect of niacin in a controlled hemorrhagic shock model in the rat and mouse. It is believed that the knowledge gained from this study will improve treatment strategies in prolonged field care (PFC) and prolonged damage control resuscitation (DCR).

### Example 2: Methods and Compositions for Modulating Hemostasis and Cellular Homeostasis Simultaneously by Intramuscular Treatment to Improve Survival Following Noncompressible Hemorrhage

Rats were subjected to non-compressible hemorrhage by liver resection and five minutes after injury, a combination of niacin (10 mg/Kg) and tranexamic acid (100 mg/Kg) were given in 200 ul volume into gastrocnemius muscle. Survival was monitored for 6 hours after the treatment

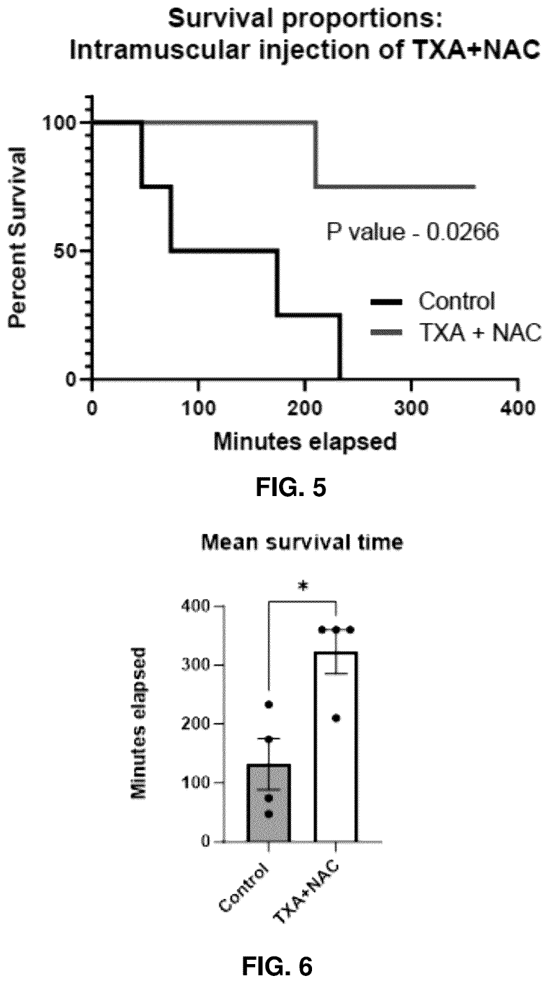

Results are shown in FIGS. 5 and 6.

Those skilled in the art will recognize, or be able to ascertain using no more than routine experimentation, many equivalents to the specific forms of the method and compositions described herein. Such equivalents are intended to be encompassed by the following claims.

I claim:

1. A method of treating a subject with a hemorrhage and/or hemorrhagic shock comprising administering the subject an effective amount of niacin or niacin-related compound and tranexamic acid to reduce one or more symptoms of the hemorrhage.

2. The method of claim 1, wherein the one or more symptoms include death, loss of homeostasis, rise in arterial blood pressure, rise in serum lactate level, and/or rise in blood pH.

3. The method of claim 1, wherein the niacin or niacin-related compound and tranexamic acid are administered in the same pharmaceutical composition.

4. The method of claim 1, wherein the niacin or niacin-related compound and tranexamic acid are in different pharmaceutical compositions.

5. The method claim 1, wherein the niacin or niacin-related compound and tranexamic acid are administered by the same route.

6. The method of claim 1, wherein the niacin or niacin-related compound and tranexamic acid are administered by different routes.

7. The method of claim 1, wherein the niacin or niacin-related compound and tranexamic acid are administered concomitantly, simultaneously, or sequentially.

8. The method of claim 1, wherein niacin or niacin-related compound and tranexamic acid are administered contemporaneously.

9. The method of claim 1, wherein the niacin or niacin-related compound and tranexamic acid are administered between 0-30 minutes apart or any subrange or specific value there between.

10. The method of claim 1, wherein the niacin or niacin-related compound and tranexamic acid are administered once.

11. The method of claim 1, wherein the niacin or niacin-related compound and tranexamic acid are administered two or more times 0.5-5 hours, optionally 1-3 hours, apart or any subrange or specific value there between.

12. The method of claim 1, wherein tranexamic acid is administered at a dosage of 5-100 mg/Kg body weight and/or the niacin or niacin-related compound is administered at 2-15 mg/Kg body weight.

13. The method of claim 1, wherein the niacin or niacin-related compound and/or tranexamic acid are administered by intramuscular injection.

14. The method of claim 1, wherein administration of the combination of niacin or niacin-related compound and tranexamic acid achieves a result greater than administering to the subject niacin or niacin-related compound alone and/or tranexamic acid alone.

15. The method of claim 1, wherein administration of the combination of niacin or niacin-related compound and tranexamic acid achieves a result that is additive or more than additive of the result obtained when administering to the subject niacin or niacin-related compound alone and tranexamic acid alone.

16. The method of claim 15, wherein the result is increased survival time and/or reduced, prevented, or reversed loss of homeosis optionally selected from reduced, prevented, and/or reversed a rise in mean arterial blood pressure; reduced, prevented, and/or reversed rise in serum lactate; reduced, prevented, and/or reversed rise blood pH; or a combination thereof.

17. The method of claim 1, wherein niacin or niacin-related compound and tranexamic acid are administered in further combination with one or more other treatments, optionally selected from controlling bleeding, replacing fluids, administering other medications optionally vasopressors.

18. The method of claim 1, wherein the subject is not currently treated and/or the results are independent of fluid resuscitation.

19. A kit comprising niacin or niacin-related compound and tranexamic acid, wherein the niacin or niacin-related compound and tranexamic acid are in the same or separate containers.

20. The kit of claim 19, comprising a syringe, optionally an autoinjector syringe, optionally loaded with an effective amount of the niacin or niacin-related compound and tranexamic acid.
